(19) United States
(12) Patent Application Publication
(10) Pub. No.: US 2022/0354200 A1
(43) Pub. Date: Nov. 10, 2022

(54) THERAPY GARMENT FOR DELIVERING AROMATHERAPY AND MEDICINAL BENEFITS AND METHODS OF MAKING AND USING THE SAME

(71) Applicant: Logan Rodriguez, Davenport, FL (US)

(72) Inventor: Logan Rodriguez, Davenport, FL (US)

(21) Appl. No.: 17/723,546

(22) Filed: Apr. 19, 2022

Related U.S. Application Data
(63) Continuation-in-part of application No. 63/185,393, filed on May 7, 2021.

(51) Int. Cl. A41D 27/20; A61K 9/00; A41D 1/04; A41B 1/00; A41D 1/06

(52) U.S. Cl. CPC A41D 27/20; A61K 9/007; A41D 1/04; A41B 1/00; A41D 1/06; A41B 2400/32; A41B 2400/36; A41D 2400/32; A41D 2400/36

(57) ABSTRACT

Systems, methods, and other embodiments associated with an aromatherapy delivery garment or a medicinal delivery garment. In one embodiment, the aromatherapy delivery garment includes a garment, a pocket assembly operatively connected to the garment, and an aromatherapy benefit delivery system located within the pocket assembly for providing an aromatherapy benefit to a wearer of the garment. In another embodiment, the medicinal benefit delivery garment includes a garment having at least one opening, a medicinal benefit delivery system located on a wearer of the garment for providing a medicinal benefit to a wearer of the garment, wherein the medicinal benefit delivery system is located within the opening, and a pocket assembly operatively connected to the garment, wherein the pocket assembly is located over the opening and the medicinal benefit delivery system in order to conceal the opening and the medicinal benefit delivery system.

### CROSS-REFERENCE TO RELATED APPLICATIONS

This application is a continuation-in-part of U.S. Provisional Patent Application 63/185,393, filed on May 7, 2021, the disclosure of which is hereby incorporated by reference in its entirety to provide continuity of disclosure to the extent such a disclosure is not inconsistent with the disclosure herein.

### FIELD OF THE INVENTION

The present invention relates to the combination of a garment and a pocket which, when used in conjunction with a benefit delivery device (such as a bag, pouch, sachet, pad, or any other form) can help deliver the benefits (aromatherapeutic and/or medicinal) of a device's contents to the wearer of the garment.

To accomplish these goals, in one embodiment, the garment has a strategically placed pocket(s) to allow for the diffusion of aromas and/or change of temperature of an area surrounding the pocket(s). Alternatively, the combination of the garment and pocket will allow for an unseen opening on the garment, which is covered by the pocket, and adjacent to the garment wearer to allow for the delivery device to have contact with the wearer's skin in order to deliver medicinal benefits to the wearer.

### BACKGROUND OF THE INVENTION

Many people suffer from asthma, allergies, and other ailments that modern medicine alone sometimes cannot alleviate. As a compliment to medical therapies, many people use alternatives such aromatherapy and skin contact/absorption solutions to help with their symptoms.

In the case of aromatherapy, the problem is that, without a diffuser, a person cannot effectively receive the desired benefits. Essential oils can be applied directly to skin exist but fade easily without constant reapplication. This is not convenient in real life when going about daily life.

It is the purpose of this invention to fulfill these and other needs in the prior art in a manner more apparent to the skilled artisan once given the following disclosure.

The preferred therapy garment, according to various embodiments of the present invention, offer the following advantages: ease of use; the ability to take all of the benefits of aromatherapy and medicinal therapy wherever you go comfortably and without even thinking about it; the elimination of messy liquids that could spill or stain clothing; lightness in weight; the elimination of a bulky garment; the ability to be able to deliver a variety of benefits, health related or otherwise, that require a proximity of a benefit delivery system to any part of the wearer's body that is to be covered with an article of clothing; the ability of the garment to not become loose or fall off; the elimination of having to attach the benefit to the wearer's neck or arm; the ability to separate the garment from the benefit delivery system; and the elimination of scent transference to other garments. In fact, in many of the preferred embodiments, these advantages are optimized to an extent that is considerably higher than heretofore achieved in prior, known systems and methods for providing a medical benefit to a person.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic illustration of a garment having a strategically placed, vented pocket for housing a benefit delivery system, constructed according to the present invention;

FIG. 2a is a schematic illustration of another garment having a strategically placed, vented pocket for housing a benefit delivery system, constructed according to the present invention;

FIG. 2b is a schematic illustration of another garment having a strategically placed, vented pocket for housing a benefit delivery system, constructed according to the present invention

FIG. 2c-2e are schematic illustrations of the pocket assembly including a vented pocket and a pocket closure system, constructed according to the present invention;

FIG. 2f is a schematic illustration of various benefit delivery systems that are capable of being located within the pocket assembly, according to the present invention;

FIG. 3a is a schematic illustration of a garment having a proximity-based delivery of the desired benefit, constructed according to the present invention;

FIG. 3b is another schematic illustration of the garment having a proximity-based delivery of the desired benefit system using a pocket, constructed according to the present invention;

FIG. 3c is a schematic illustration of the garment having the proximity-based delivery of the desired benefit system, constructed according to the present invention;

FIG. 3d is a schematic illustration of the benefit delivery system being located underneath a pocket for the garment having the proximity-based delivery of the desired benefit system, according to the present invention;

FIGS. 3e-3g are schematic illustrations of the pocket assembly including a pocket and a pocket closure system, constructed according to the present invention; and

FIGS. 3h and 3i are schematic illustrations of the garment having the proximity-based delivery of the desired benefit system, constructed according to the present invention.

### DETAILED DESCRIPTION OF INVENTION, EMBODIMENTS

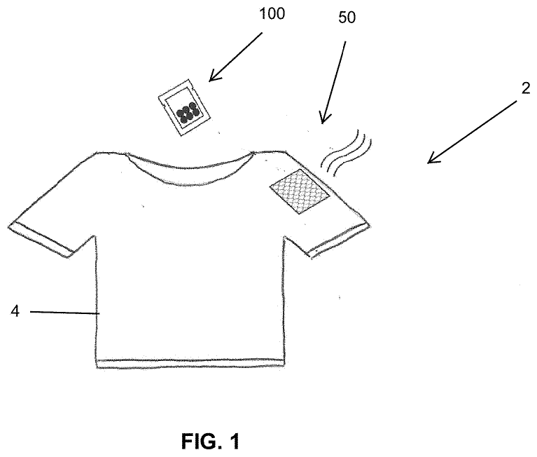

In order to address the shortcomings of the prior known systems and methods for providing a medical benefit to a person, reference is now made to FIG. 1 where there is illustrated a garment system 2 having a strategically placed, vented pocket assembly 50 for housing a benefit delivery system 100. Garment system 2 includes, in part, garment 4 (such as a shirt). It is to be understood that while a shirt is shown, the garment 4 could also be pants or other similar clothing items as long as the as the pocket system 50 and the benefit delivery system 100 can be located on the garment 4.

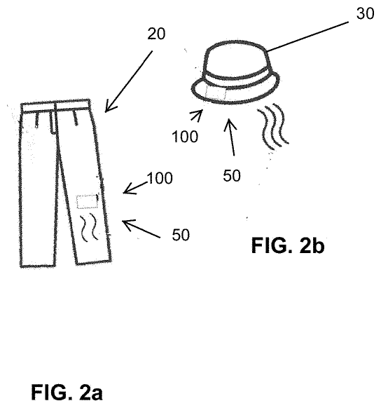

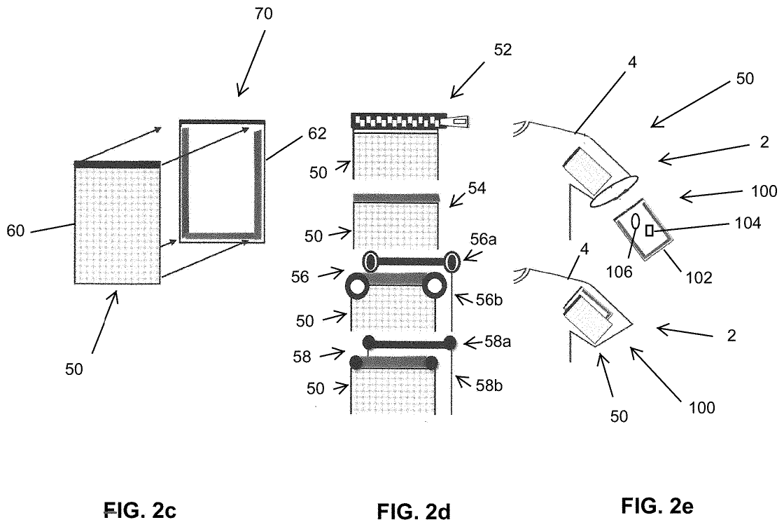

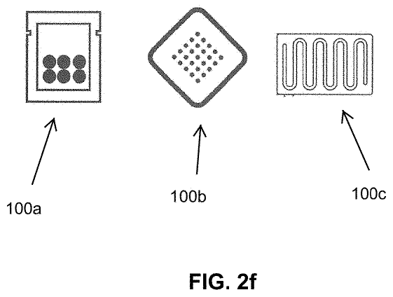

In particular, as shown in FIGS. 2a and 2b, pants 20 can be constructed to include a strategically placed, vented pocket assembly 50 for housing a benefit delivery system 100. Also, hat 30 can be constructed to include a strategically placed, vented pocket assembly 50 for housing a benefit delivery system 100.

With respect to FIGS. 2c-2e, there is illustrated another embodiment of a garment system 2 having a strategically placed, vented pocket assembly 50 for housing a benefit delivery system 100. As discussed above, garment system 2 includes, in part, garment 4 (such as a shirt). Preferably garment 4 is constructed of any suitable flexible clothing material such as cotton, wool, polyester, a blend of cotton and polyester or the like. It is also within the scope of this invention that the garment 4 can be constructed of one or more alternative materials like alternative fabrics. These alternative fabric materials can include a mix of both regular and stretch fabric threads.

In one embodiment, garment system 2 is the combination of a garment 4, a strategically placed pocket system 50, and a benefit delivery system 100 (such as a container 102 with inclusions 104 (FIG. 2e)) that delivers health related or otherwise benefits (aromatic and/or medicinal) to the wearer while on the go.

For an aromatherapy application, in one embodiment, the benefit delivery system 100 includes a container 102 (such as a sachet) with aromatherapy inclusions 104 that will be placed inside the pocket system 50 (FIG. 2e). In one embodiment, the container 102 mixes the aromatherapy inclusions 104 with salts 106 (FIG. 2e), so that the benefits last longer. In another embodiment, both the pocket assembly 50 and the container 102 will be vented in a way so that the scents from the aromatherapy inclusions 104 and the salts 106 can reach the olfactory system of the wearer. A unique aspect of the present invention is that by using a container 102 (such as a sachet), garment stains from the aromatherapy inclusions 104 and the salts 106 can be substantially eliminated.

As shown in FIGS. 2c-2e, the design of the pocket assembly 50 will allow for a simple way of inserting the benefit delivery system 100 into the pocket assembly 50, thereby making garment system 2 accessible to people with disabilities, arthritis, and the elderly which is a practical application of the present invention. In particular, in one embodiment, the pocket assembly 50 will have a closure assembly, including but not limited to, a zipper assembly 52 (FIG. 2d), Velcro® assembly 54 (FIG. 2d), a snap fastener assembly 56 (FIG. 2d), a wearable magnet assembly 58 (FIG. 2d) or any type of closable assembly that prevents the benefit delivery system 100 from falling out of the pocket assembly 50. In this manner, once the benefit delivery system 100 is placed within pocket assembly 50, the closure assembly 52, 54, 56, or 58 is activated in order to removably retain benefit delivery system 100 within pocket assembly 50.

It is to be understood that in another embodiment, the pocket assembly 50 can also be devoid of any type of closure assembly 52, 54, 56, or 58 for easier access.

Furthermore, in one embodiment, the pocket assembly 250 can be made from a mesh-like material 60 (FIG. 2c) that can be attached to a pocket wall layer 62 by attaching techniques such as sewing, hook and loop fasteners, magnets, snap fasteners or the like. However, it is within the scope of the present invention that the pocket assembly 50 includes one or more alternative materials and fabrics, as discussed earlier. It is to be understood that whatever material is used for pocket assembly 50, the material should allow for the scents from the aromatherapy inclusions 104 and the salts 106 to reach the olfactory system of the wearer.

Another unique aspect of the present invention is that the garment system 2 includes a strategically placed, affixed pocket assembly 50 or the pocket assembly 50 that can be removed and be placed securely anywhere on the garment 4 the user prefers by means of a holding mechanism 70 (FIG. 2e) that securely holds the pocket assembly 50 in place on the garment 4. For example, as shown in FIG. 2d, in one embodiment, snap fasteners 56a would be conventionally attached to garment 4 and snap fasteners 56b which are attached to pocket assembly 50 could interact with snap fasteners 56a in order to secure pocket assembly 50 to garment 4. Also, as shown in FIG. 2d, in one embodiment, magnet fasteners 58a would be conventionally attached to garment 4 and magnet fasteners 58b which are attached to pocket assembly 50 could interact with magnet fasteners 58a in order to secure pocket assembly 50 to garment 4.

Another unique aspect of the present invention is that even though the benefit delivery system 100 is integrated into the garment 4, it should be understood that the benefit delivery system 100 could be provided as part of an alternate garment such as a scarf, shawl, wrap, cloak, hat, shirt, gloves, sweater, jacket, etc. as long as the location of the benefit delivery system 100 on the alternate garment allows for the scents from the aromatherapy inclusions 104 and the salts 106 to reach the olfactory system of the wearer, as shown in FIGS. 1, 2a, 2b, and 2e.

A still another unique aspect of the present invention is that beyond an aromatherapy application, the benefit delivery system 100 used in conjunction with the garment 4 and pocket assembly 50 applies to other applications, including but not limited to, prescription and over the counter medicines, therapies, fragrances and/or storage of any material suited to fit in the pocket assembly 50.

With respect to FIG. 2f, various benefit delivery systems 100 are shown. In particular, the benefit delivery system 100 can be in the form of a sachet 100a, a medicinal or thermal patch 100b, and/or an electronic pulse or other similar type of device 100c. An important consideration of the type of benefit delivery system 100 being utilized being that the benefit delivery system 100 should provide an aromatic or other similar medicinal benefit to the wearer.

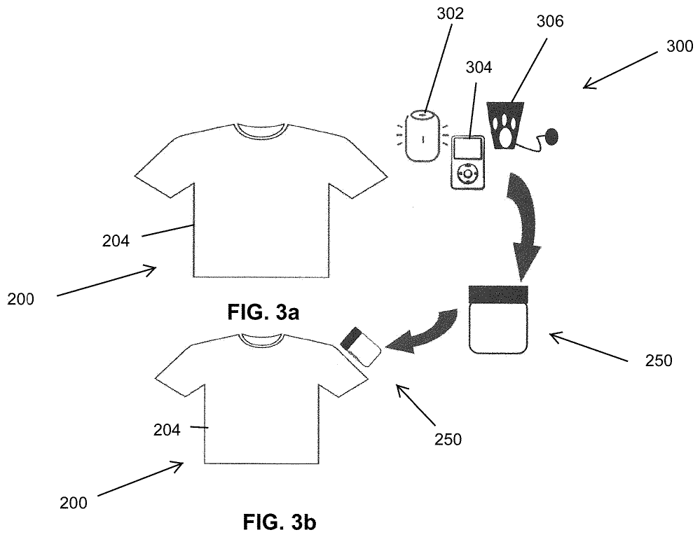

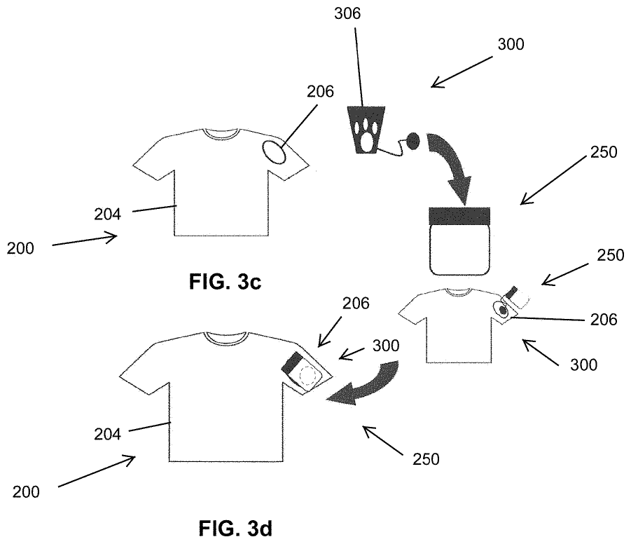

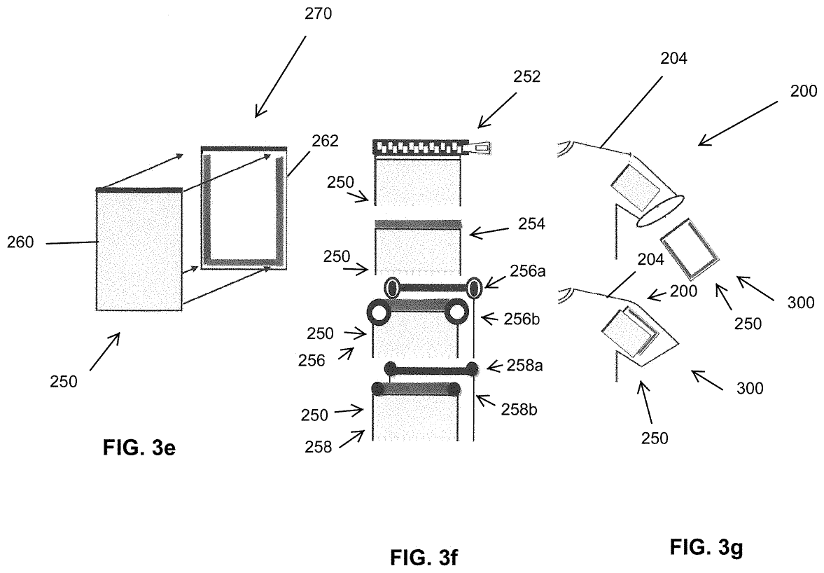

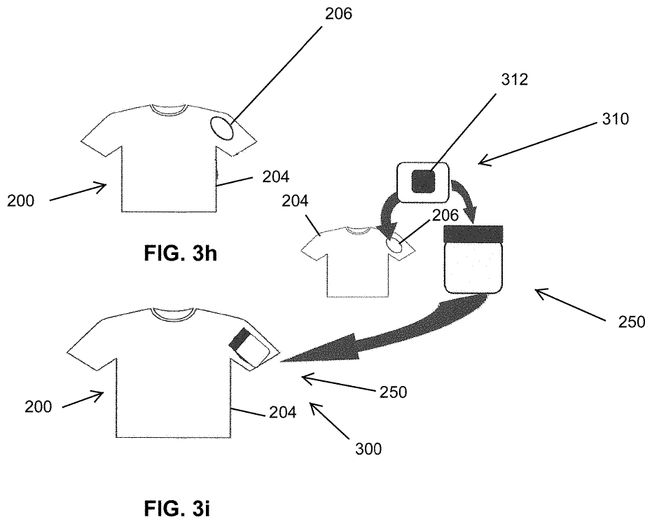

With respect to FIGS. 3a-3d, there is illustrated a garment system 200 having a proximity-base delivery system 300 of the desired benefit. In one embodiment, garment system 200 includes, in part, garment 204 (such as a shirt). It is to be understood that while a shirt is shown, the garment 204 could also be pants or other similar clothing items as long as the as the pocket system 250 and the benefit delivery system 300 can be located on the garment 204.

With respect to FIGS. 3a-3d, there is illustrated another embodiment of a garment system 200 having a strategically placed, pocket assembly 250 for containing/covering/concealing a benefit delivery system 300. As discussed above, garment assembly 200 includes, in part, garment 204 (such as a shirt). Preferably garment 204 is constructed of any suitable flexible clothing material such as cotton, wool, polyester, a blend of cotton and polyester or the like. It is also within the scope of this invention that the garment 204 can be constructed of one or more alternative materials like alternative fabrics. The alternative fabric materials can include a mix of both regular and stretch fabric threads.

In one embodiment, garment system 200 is the combination of a garment 204, a strategically placed pocket system 250, and a benefit delivery system 300 (such as a speaker 302, mobile communication device 304 and/or sensor 306 (FIG. 3a)) that can be used to provide medical/health related information between the wearer and a medical provider (not shown), while on the go. For example, the mobile communication device 304 and sensor 306 can be used to provide information to a medical provider regarding the benefit delivery system 300 and the wearer of the garment 204. The speaker 302 can then be used to alert the wearer that the wearer should check the benefit delivery system 300 for a possible communication on the mobile communication device 304 from the medical provider, for example.

As further shown in FIG. 3b, the pocket system 250 can be attached to the garment 204, as discussed above. In this manner, the benefit delivery system 300 can be carried around by the wearer so that there can be a continuous flow of medical information between the wearer and the medical provider.

With respect to FIGS. 3c and 3d, in one embodiment, the benefit delivery system 300 includes sensor 306 that will be located on the wearer and subsequently covered over/concealed by the pocket system 250. It is to be understood that sensor 306 should come into contact with the wearer's skin so that the sensor 306 can interact with the wearer and provide information to the medical provider about the wearer. In another embodiment, the sensor 306 is placed on the wearer through the opening 206 in the garment 204. The pocket system 250 is then located over the sensor 306 and the opening 206 in order to cover over/conceal the sensor 306 of the benefit delivery system 300 and the opening 206.

As shown in FIGS. 3e-3g, the design of the pocket assembly 250 will allow for a simple way of inserting the benefit delivery system 300 into the pocket assembly 250, thereby making garment system 200 accessible to people with disabilities, arthritis, and the elderly which is a practical application of the present invention. In particular, in one embodiment, the pocket assembly 250 will have a closure assembly, including but not limited to, a zipper assembly 252 (FIG. 3f), Velcro® assembly 254 (FIG. 3f), a snap fastener assembly 256 (FIG. 3f), a wearable magnet assembly 258 (FIG. 3f) or any type of closable assembly that prevents the benefit delivery system 300 from falling out of the pocket assembly 250. In this manner, once the benefit delivery system 300 is placed within pocket assembly 250, the closure assembly 252, 254, 256, or 258 is activated in order to removably retain benefit delivery system 300 within pocket assembly 250.

Furthermore, in one embodiment, the pocket assembly 250 can be made from a material 260 (FIG. 3e) that can be attached to a pocket wall layer 262 by attaching techniques such as sewing, hook and loop fasteners, magnets, snap fasteners or the like. However, it is within the scope of the present invention that the pocket assembly 250 includes one or more alternative materials and fabrics, as discussed earlier. It is to be understood that whatever material is used for pocket assembly 250, the material should allow for the benefit delivery system 300 to properly interact with the wearer.

Another unique aspect of the present invention is that the garment system 200 includes a strategically placed, affixed pocket assembly 250 or the pocket assembly 250 can be removed and be placed securely anywhere on the garment 204 the user prefers by means of a holding mechanism 270 (FIG. 3e) that securely holds the pocket assembly 250 in place on the garment 204. For example, as shown in FIG. 3f, in one embodiment, snap fasteners 256a would be conventionally attached to garment 204 and snap fasteners 256b which are attached to pocket assembly 250 could interact with snap fasteners 256a in order to secure pocket assembly 250 to garment 204. Also, as shown in FIG. 3f, in one embodiment, magnet fasteners 258a would be conventionally attached to garment 204 and magnet fasteners 258b which are attached to pocket assembly 250 could interact with magnet fasteners 258a in order to secure pocket assembly 250 to garment 204.

With respect to FIGS. 3h and 3i, in one embodiment, the benefit delivery system 300 includes adhesive bandage 310 with a medicament 312 that will be located on the wearer and subsequently covered over/concealed by the pocket system 250. It is to be understood that the medicament 312 should come into contact with the wearer's skin so that the medicament 312 can provide the proper medicinal benefits to the wearer. In another embodiment, the benefit delivery system 300 is placed on the wearer through the opening 206 in the garment 204. The pocket system 250 is then located over the benefit delivery system 300 and the opening 206 in order to cover over/conceal the benefit delivery system 300 and the opening 206.

A unique aspect of the present invention is that the garment system 200 includes a strategically placed, affixed pocket assembly 250 or the pocket assembly 250 that can be removed and be placed securely anywhere on the garment 204 the user prefers by means of a holding mechanism that securely holds the pocket assembly 250 in place over opening 206 on the garment 204, as discussed above. In this manner, the pocket assembly 250 can be located over any openings 206 that are located on garment 204. If needed, a plurality of pocket assemblies 250 may need to be utilized in order to cover over/conceal a plurality of benefit delivery systems 300 and openings 206.

Another unique aspect of the present invention is that even though the benefit delivery system 300 is associated with the illustrated garment 204, it should be understood that the benefit delivery system 300 could be provided as part of an alternate garment such as a scarf, shawl, wrap, cloak, hat, shirt, gloves, sweater, jacket, etc. as long as the location of the benefit delivery system 300 on the alternate garment allows for the medicinal benefits of the medicament 304 to properly interact with the wearer.

A still another unique aspect of the present invention is that beyond a medicinal application, the benefit delivery system 300 used in conjunction with the garment 204 and pocket assembly 250 applies to any other applications, including but not limited to, prescription and over the counter medicines, therapies, fragrances and/or storage of any material.

### Construction of Garment Systems

During the construction of the garment system 2, attention is directed to FIGS. 1 and 2a-2f. In particular, in one embodiment, the pocket assembly 50 is attached to a sleeve of the garment 4. As discussed earlier, preferably garment 4 is constructed of any suitable flexible clothing material such as cotton, wool, polyester, a blend of cotton and polyester or the like. It is also within the scope of this invention that the garment 4 can be constructed of one or more alternative materials like alternative fabrics. The alternative fabric materials can include a mix of both regular and stretch fabric threads.

In another embodiment, the garment 4 could also utilize pants or other similar clothing items as long as the as the pocket system 50 and the benefit delivery system 100 can be located on the clothing item (FIGS. 2a and 2b). Furthermore, the pocket assembly 50 can be located on number of locations on the garment 4 as long as the scents from the aromatherapy inclusions 104 and the salts 106 or other medicinal benefits can reach the olfactory system of the wearer. Finally, a plurality of pocket assemblies 50 can be located on the garment 4 in order to house a number of benefit delivery systems 100. In this manner, the different (or the same) aromatherapy inclusions 104 and the salts 106 or other medicinal benefits may be located on the garment 4 in order to allow an even greater amount of the aromatherapy inclusions 104 and the salts 106 or other medicinal benefits to reach the olfactory system of the wearer.

As discussed above, in one embodiment, the benefit delivery system 100 includes a container 102 (such as a sachet) with aromatherapy inclusions 104 that will be placed inside the pocket system 50 (FIG. 2e). In another embodiment, the benefit delivery system 100 can include a patch 101b and/or an electronic device 100c, as discussed above. In one embodiment, the container 102 mixes the aromatherapy inclusions 104 with salts 106, so that the benefits last longer. In another embodiment, both the pocket assembly 50 and/or the container 102 will be vented in a way so that the scents from the aromatherapy inclusions 104 and the salts 106 can reach the olfactory system of the wearer. In another embodiment, the pocket assembly 50 will be vented to allow the patch 100b and/or the electronic device 100c to properly interact with the wearer. A unique aspect of the present invention is that by using a container 102 (such as a sachet), garment stains from the aromatherapy inclusions 104 and the salts 106 can be substantially eliminated.

With respect to pocket assembly 50, the design of the pocket assembly 50 will allow for a simple way of inserting the benefit delivery system 100 into the pocket assembly 50, thereby making garment system 2 accessible to people with disabilities, arthritis, and the elderly which is a practical application of the present invention. In particular, in one embodiment, the pocket assembly 50 will have a closure assembly, including but not limited to, a zipper assembly 52 (FIG. 2d), Velcro® assembly 54 (FIG. 2d), a snap fastener assembly 56 (FIG. 2d), a wearable magnet assembly 58 (FIG. 2d) or any type of closable assembly that prevents the benefit delivery system 100 from falling out of the pocket assembly 50. In this manner, once the benefit delivery system 100 is placed within pocket assembly 50, the closure assembly 52, 54, 56, or 58 is activated in order to removably retain benefit delivery system 100 within pocket assembly 50.

As discussed above, in another unique aspect of the present invention is that the garment system 2 includes a strategically placed, affixed pocket assembly 50 or the pocket assembly 50 that can be removed and be placed securely anywhere on the garment 4 the user prefers by means of a holding mechanism 60 (FIG. 2c) that securely holds the pocket assembly 50 in place on the garment 4. Furthermore, as shown in FIG. 2d, in one embodiment, snap fasteners 56a would be conventionally attached to garment 4 and snap fasteners 56b which are attached to pocket assembly 50 could interact with snap fasteners 56a in order to secure pocket assembly 50 to garment 4. Also, as shown in FIG. 2d, in one embodiment, magnet fasteners 58a would be conventionally attached to garment 4 and magnet fasteners 58b which are attached to pocket assembly 50 could interact with magnet fasteners 58a in order to secure pocket assembly 50 to garment 4

Another unique aspect of the present invention is that even though the benefit delivery system 100 is integrated into the garment 4, it should be understood that the benefit delivery system 100 could be provided as part of an alternate garment such as a scarf, shawl, wrap, cloak, hat, shirt, gloves, sweater, jacket, etc. as long as the location of the benefit delivery system 100 on the alternate garment allows for the scents from the aromatherapy inclusions 104 and the salts 106 or other medicinal benefits to reach the olfactory system of the wearer.

A still another unique aspect of the present invention is that beyond an aromatherapy application, the benefit delivery system 100 used in conjunction with the garment 4 and pocket assembly 50 applies to any other applications, including but not limited to, prescription and over the counter medicines, therapies, fragrances and/or storage of any material suited to fit in the pocket assembly 50.

It is to be understood that in another embodiment, the pocket assembly 50 can also be devoid of any type of closure assembly 52, 54, 56, or 58 for easier access.

With respect to FIGS. 3a-3i, there is illustrated a garment system 200 having a proximity-based delivery system 300 of the desired benefit. During the construction of this embodiment, the garment system 200 will include a strategically placed (removably or permanently), pocket assembly 250 for containing/covering over/concealing a benefit delivery system 300. Garment system 200 includes, in part, garment 204 (such as a shirt). It is to be understood that while a shirt is shown, the garment 204 could also be pants or other similar clothing items as long as the as the pocket system 250 can be located on the garment 204 and the garment provides an opening 206.

Furthermore, the pocket assembly 250 can be located on a number of locations on the garment 204 as long as the benefit delivery system 300 can properly interact with the wearer. Also, a plurality of pocket assemblies 250 can be located on the garment 204 in order to conceal a number of benefit delivery systems 300. In this manner, the different (or the same) benefit delivery systems 300 may be located on the wearer to allow an even greater amount of the medical benefit to the wearer.

In one embodiment, a benefit delivery system 300 may include a speaker 302, mobile communication device 304, and/or sensor 306 (FIG. 3a). These components can be used to provide medical/health related information between the wearer and a medical provider, while on the go. For example, the mobile communication device 304 and sensor 306 can be used to provide information to a medical provider regarding the benefit delivery system 300 and the wearer of the garment 204. The speaker 302 can then be used to alert the wearer that the wearer should check the benefit delivery system 300 for a possible communication on the mobile communication device 304 from the medical provider, for example.

As further shown in FIG. 3b, the pocket system 250 can be attached to the garment 204, as discussed above. In this manner, the benefit delivery system 300 can be carried around by the wearer so that there can be a continuous flow of medical information between the wearer and the medical provider.

With respect to FIGS. 3c and 3d, in one embodiment, the benefit delivery system 300 may include sensor 306 that will be located on the wearer and subsequently contained/covered over/concealed by the pocket system 250. It is to be understood that sensor 306 should come into proximity or contact with the wearer's skin so that the sensor 306 can interact with the wearer and provide information to the medical provider about the wearer. In another embodiment, the sensor 306 is placed on the wearer through the opening 206 in the garment 204. The pocket system 250 is then located over the sensor 306 and the opening 206 in order to cover over/conceal the sensor 306 of the benefit delivery system 300 and the opening 206.

As shown in FIGS. 3f and 3g, the design of the pocket assembly 250 will allow for a simple way of inserting the benefit delivery system 300 into the pocket assembly 250, thereby making garment system 200 accessible to people with disabilities, arthritis, and the elderly which is a practical application of the present invention. In particular, in one embodiment, the pocket assembly 250 will have a closure assembly, including but not limited to, a zipper assembly 252 (FIG. 3f), Velcro® assembly 254 (FIG. 3f), a snap fastener assembly 256 (FIG. 3f), a wearable magnet assembly 258 (FIG. 3f) or any type of closable assembly that prevents the benefit delivery system 300 from falling out of the pocket assembly 250. In this manner, once the benefit delivery system 300 is placed within pocket assembly 250, the closure assembly 252, 254, 256, or 258 is activated in order to removably retain benefit delivery system 300 within pocket assembly 250.

Furthermore, in one embodiment, the pocket assembly 250 can be made from a material 260 (FIG. 3e) that can be attached to a pocket wall layer 262 by attaching techniques such as sewing, hook and loop fasteners, magnets or snap fasteners. However, it is within the scope of the present invention that the pocket assembly 250 includes one or more alternative materials and fabrics, as discussed earlier. It is to be understood that whatever material is used for pocket assembly 250, the material should allow for the benefit delivery system 300 to properly interact with the wearer.

Another unique aspect of the present invention is that the garment system 200 includes a strategically placed, affixed pocket assembly 250 or the pocket assembly 250 that can be removed and be placed securely anywhere on the garment 204 the user prefers by means of a holding mechanism 270 (FIG. 3e) that securely holds the pocket assembly 250 in place on the garment 204. For example, as shown in FIG. 3f, in one embodiment, snap fasteners 256a would be conventionally attached to garment 204 and snap fasteners 256b which are attached to pocket assembly 250 could interact with snap fasteners 256a in order to secure pocket assembly 250 to garment 204. Also, as shown in FIG. 3f, in one embodiment, magnet fasteners 258a would be conventionally attached to garment 204 and magnet fasteners 258b which are attached to pocket assembly 250 could interact with magnet fasteners 258a in order to secure pocket assembly 250 to garment 204.

For a medicament delivery application, in one embodiment, the benefit delivery system 300 includes adhesive bandage 310 with a medicament 312 (FIGS. 3h and 3i) that will be placed on the wearer. In one embodiment, the garment 204 includes an opening 206 in which the benefit delivery system 300 can be located adjacent to the skin of the wearer such that the pocket assembly 250 can conceal the benefit delivery system 300. In another embodiment, the benefit delivery system 300 is placed on the wearer through the opening 206 in the garment 204. The pocket system 250 is then located over the benefit delivery system 300 and the opening 206 in order to conceal the benefit delivery system 300 and the opening 206.

With respect to pocket assembly 250, the design of the pocket assembly 250 will allow for a simple way of locating the benefit delivery system 300 on the wearer, thereby making garment system 200 accessible to people with disabilities, arthritis, and the elderly which is a practical application of the present invention.

Another unique aspect of the present invention is that even though the benefit delivery system 300 is associated with the garment 204, it should be understood that the benefit delivery system 300 could be provided as part of an alternate garment such as a scarf, shawl, wrap, cloak, hat, shirt, gloves, sweater, jacket, etc. as long as the location of the benefit delivery system 300 on the alternate garment allows for the medicinal benefits of the medicament 304 to properly interact with the wearer.

A still another unique aspect of the present invention is that beyond a medicinal application, the benefit delivery system 300 used in conjunction with the garment 204 and pocket assembly 250 applies to any other applications, including but not limited to, prescription and over the counter medicines, therapies, fragrances.

Furthermore, in one embodiment, the pocket 252 can be made from a solid or mesh-like material. However, it is within the scope of the present invention that the pocket 252 may include one or more alternative materials and fabrics. It is to be understood that whatever material is used for pocket 252, the material should allow for the medicinal benefits of the medicament 304 or other medicinal benefits to properly interact with the wearer.

### Using Garment Systems

With respect to using the garment system 2, attention is directed to FIGS. 1 and 2a-2f. In particular, pocket assembly 50 is attached (removably or permanently) to the garment 4. If the pocket assembly 50 is permanently attached to garment 4, conventional attachment techniques such as sewing, adhesives, fasteners or the like are used to attach the pocket assembly 50 to the garment 4. As discussed earlier, a plurality of pocket assemblies 50 can be attached to garment 4.

Once the pocket assembly 50 is attached to garment 4, the benefit delivery system 100 can be placed within the pocket assembly 50, as discussed earlier. In particular, in one embodiment, the pocket assembly 50 will have a closure assembly, including but not limited to, a zipper assembly 52 (FIG. 2d), Velcro® assembly 54 (FIG. 2d), a snap fastener assembly 56 (FIG. 2d), a wearable magnet assembly 58 (FIG. 2d) or any type of closable assembly that prevents the benefit delivery system 100 from falling out of the pocket assembly 50. In this manner, once the benefit delivery system 100 is placed within pocket assembly 50, the closure assembly 52, 54, 56, or 58 is activated in order to removably retain benefit delivery system 100 within pocket assembly, thereby making garment system 2 accessible to people with disabilities, arthritis, and the elderly which is a practical application of the present invention.

As discussed above, another unique aspect of the present invention is that the garment system 2 includes a strategically placed, affixed pocket assembly 50 or the pocket assembly 50 can be removed and be placed securely anywhere on the garment 4 the user prefers by means of a holding mechanism 60 (FIGS. 2a and 2b) that securely holds the pocket assembly 50 in place on the garment 4. For example, as discussed above, fasteners such as hook and loop fasteners (such as Velcro®) or other fastener types such as snaps, adhesive backing, etc. may be used.

After the benefit delivery system 100 is placed within the pocket assembly 50, the scents from the aromatherapy inclusions 104 and the salts 106 or other medicinal benefits will escape out of the pocket 52 and reach the olfactory system of the wearer in order to provide the necessary aromatherapy benefits to the wearer. If a patch 100b and or electronic device 100c is located within pocket assembly 50, then pocket assembly 50 will allow these components to properly interact with the wearer and the medical provider, as discussed above.

Another unique aspect of the present invention is that even though the benefit delivery system 100 is integrated into the illustrated garment 4, it should be understood that the benefit delivery system 100 could be provided as part of an alternate garment such as a scarf, shawl, wrap, cloak, hat, shirt, gloves, sweater, jacket, etc. as long as the location of the benefit delivery system 100 on the alternate garment allows for the scents from the aromatherapy inclusions 104 and the salts 106 or other medicinal benefits to reach the olfactory system of the wearer.

A still another unique aspect of the present invention is that beyond an aromatherapy application, the benefit delivery system 100 used in conjunction with the garment 4 and pocket assembly 50 applies to any other applications, including but not limited to, prescription and over the counter medicines, therapies, fragrances and/or storage of any material suited to fit in the pocket assembly 50.

With respect to using the garment system 200, attention is directed to FIGS. 3a-3i. In particular, the pocket assembly 250 is attached (removably or permanently) on garment 204 so that pocket assembly 250 is located on garment 204 and/or over opening 206 on garment 204. The benefit delivery system 300 is located on the wearer and positioned so as to be able to be located within opening 206. In another embodiment, the pocket assembly 250 is attached to garment 204 (FIGS. 3a and 3b). If the pocket assembly 250 is permanently attached to garment 204, conventional attachment techniques such as sewing, adhesives, fasteners or the like are used to attach the pocket assembly 250 to the garment 204. As discussed earlier, a plurality of pocket assemblies 250 can be attached to a plurality of openings 206 on garment 204 or directly to garment 204 (FIGS. 3a and 3b).

As discussed above, another unique aspect of the present invention is that the garment system 200 includes a strategically placed, affixed pocket assembly 250 or the pocket assembly 250 can be removed and be placed securely anywhere on the garment 204 the user prefers by means of a holding mechanism that securely holds the pocket assembly 50 in place on opening 206 the garment 204, as discussed above. For example, fasteners such as hook and loop fasteners (such as Velcro®) or other fastener types such as snaps, adhesive backing, etc. may also be used (FIG. 3f).

After the benefit delivery system 300 is placed on the wearer and within the opening 206, the pocket assembly 250 will then be placed over the benefit delivery system 300 and, optionally over the opening 206, in order to contain, cover or otherwise conceal the benefit delivery system 300 and allow the medicinal benefits of benefit delivery system 300 to interact with the wearer.

Another unique aspect of the present invention is that even though the benefit delivery system 300 is integrated into the illustrated garment 204, it should be understood that the benefit delivery system 300 could be provided as part of an alternate garment such as a scarf, shawl, wrap, cloak, hat, shirt, gloves, sweater, jacket, etc. as long as the alternate garment contains an opening 206 and the alternate garments will cover or otherwise conceal the benefit delivery system 300 and allow the medicinal benefits of benefit delivery system 300 to interact with the wearer.

A still another unique aspect of the present invention is that beyond a medicinal application, the benefit delivery system 300 used in conjunction with the garment 204 and pocket assembly 250 applies to any other applications, including but not limited to, prescription and over the counter medicines, therapies, fragrances.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

This description of the exemplary embodiments is intended to be read in connection with the figures of the accompanying drawing, which are to be considered part of the entire written description. In the description, relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. Furthermore, the term fluid/gas/liquid are meant to be used interchangeably.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety.

The applicant reserves the right to physically incorporate into this specification any and all materials and information from any such patents, publications, scientific articles, web sites, electronically available information, and other referenced materials or documents to the extent such incorporated materials and information are not inconsistent with the description herein.

The written description portion of this patent includes all claims. Furthermore, all claims, including all original claims as well as all claims from any and all priority documents, are hereby incorporated by reference in their entirety into the written description portion of the specification, and Applicant(s) reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended and those skilled in the art will recognize, that the invention embraces and is also thereby described in terms of any individual member or subgroup of members of the Markush group.

Other embodiments are within the following claims. Therefore, the patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

Other modifications and implementations will occur to those skilled in the art without departing from the spirit and the scope of the invention as claimed. Accordingly, the description hereinabove is not intended to limit the invention, except as indicated in the appended claims.

Therefore, provided herein is a new and improved therapy garment. The preferred therapy garment, according to various embodiments of the present invention, offer the following advantages: ease of use; the ability to take all of the benefits of aromatherapy and medicinal therapy wherever you go comfortably and without even thinking about it; the elimination of messy liquids that could spill or stain clothing; lightness in weight; the elimination of a bulky garment; the ability to be able to deliver a variety of benefits, health related or otherwise, that require a proximity of a benefit delivery system to any part of the wearer's body that is to be covered with an article of clothing; the ability of the garment to not become loose or fall off; the elimination of having to attach the benefit to the wearer's neck or arm; the ability to separate the garment from the benefit delivery system; and the elimination of scent transference to other garments.

In fact, in many of the preferred embodiments, these advantages of ease of use, the ability to take all of the benefits of aromatherapy and medicinal therapy wherever you go comfortably and without even thinking about it, the elimination of messy liquids that could spill or stain clothing, lightness in weight, the elimination of a bulky garment, the ability to be able to deliver a variety of benefits, health related or otherwise, that require a proximity of a benefit delivery system to any part of the wearer's body that is to be covered with an article of clothing, the ability of the garment to not become loose or fall off, the elimination of having to attach the benefit to the wearer's neck or arm, the ability to separate the garment from the benefit delivery system, and the elimination of scent transference to other garments are optimized to an extent that is considerably higher than heretofore achieved in prior, known systems and methods for providing a medical benefit to a person

I claim:

1. An aromatherapy delivery garment comprising:
a garment;
a pocket assembly operatively connected to the garment; and
an aromatherapy benefit delivery system located within the pocket assembly for providing an aromatherapy benefit to a wearer of the garment.

2. The aromatherapy delivery garment, according to claim 1, wherein the garment is further comprised of:
a shirt having a plurality of sleeves,
wherein the pocket assembly is operatively connected to at least one of the plurality of sleeves.

3. The aromatherapy delivery garment, according to claim 1, wherein the pocket assembly is further comprised of:
a pocket, wherein the benefit delivery system is located with the pocket.

4. The aromatherapy delivery garment, according to claim 3, wherein the pocket is further comprised of:
a mesh-like material.

5. The aromatherapy delivery garment, according to claim 3, wherein the pocket assembly is further comprised of:
a closure assembly operatively connected to one end of the pocket,
wherein the closure assembly includes, but is not limited to zippers, Velcro®, buttons, hooks, or snaps.

6. The aromatherapy delivery garment, according to claim 1, wherein the aromatherapy delivery garment is further comprised of:
at least one first fastener operatively connected to the garment; and
at least one second fastener operatively connected to the pocket,
wherein the at least one first fastener and the at least one second fastener interact with each other in order to removably retain the pocket assembly on the garment.

7. The aromatherapy delivery garment, according to claim 6, wherein the at least one first fastener and the at least one second fastener are further comprised of:
hook and loop fasteners or other fastener types such as snaps and adhesives.

8. A method of constructing an aromatherapy delivery garment comprising the steps of:
providing a garment;
attaching a pocket assembly to the garment; and
locating an aromatherapy benefit delivery system within the pocket assembly for providing an aromatherapy benefit to a wearer of the garment.

9. The method, according to claim 8, wherein the step of providing the garment is further comprised of:
providing a shirt having a plurality of sleeves; and
attaching the pocket assembly to at least one of the plurality of sleeves.

10. The method, according to claim 8, wherein the step of operatively attaching the pocket assembly is further comprised of:
providing a pocket, wherein the benefit delivery system is located with the pocket.

11. The method, according to claim 10, wherein the pocket is further comprised of:
a mesh-like material.

12. The method, according to claim 10, wherein the step of attaching the pocket assembly is further comprised of:
providing a closure assembly on one end of the pocket,
wherein the closure assembly includes, but is not limited to zippers, Velcro®, buttons, hooks, or snaps.

13. The method, according to claim 8, wherein the method is further comprised of the steps of:
attaching at least one first fastener to the garment; and
attaching at least one second fastener to the pocket,
wherein the at least one first fastener and the at least one second fastener interact with each other in order to removably retain the pocket assembly on the garment.

14. The method, according to claim 13, wherein the at least one first fastener and the at least one second fastener are further comprised of:
hook and loop fasteners or other fastener types such as snaps and adhesives.

15. A medicinal benefit delivery garment comprising:
a garment having at least one opening;
a medicinal benefit delivery system located on a wearer of the garment for providing a medicinal benefit to a wearer of the garment,
wherein the medicinal benefit delivery system is located within the opening; and
a pocket assembly operatively connected to the garment,
wherein the pocket assembly is located over the opening and the medicinal benefit delivery system in order to conceal the opening and the medicinal benefit delivery system.

16. The medicinal benefit delivery garment, according to claim 15, wherein the garment is further comprised of:
a shirt having a plurality of sleeves,
wherein the opening is located on at least one of the plurality of sleeves and the pocket assembly is operatively connected to the at least one of the plurality of sleeves.

17. The medicinal benefit delivery garment, according to claim 15, wherein the pocket assembly is further comprised of:
a pocket, wherein the pocket conceals the opening and the benefit delivery system.

18. The medicinal benefit delivery garment, according to claim 15, wherein the medicinal benefit delivery system is further comprised of:
an adhesive bandage; and
a medicament located on one side of the adhesive bandage,
wherein the medicament is located adjacent to the wearer.

19. The medicinal benefit delivery garment, according to claim 15, wherein the garment is further comprised of:
at least one first fastener operatively connected to the garment; and
at least one second fastener operatively connected to the pocket,
wherein the at least one first fastener and the at least one second fastener interact with each other in order to removably retain the pocket assembly on the garment.

20. The medicinal benefit delivery garment, according to claim 19, wherein the at least one first fastener and the at least one second fastener are further comprised of:
hook and loop fasteners or other fastener types such as snaps and adhesives.
